(19) United States
(12) Patent Application Publication
(10) Pub. No.: US 2006/0100447 A1
(43) Pub. Date: May 11, 2006

(54) Process for the production of y-methyl-a-methylene-y-butyrolactone from reaction of levulinic acid and hydrogen with recycle of unreacted levulinic acid followed by reaction of crude y-valerolactone and formaldehyde, both reactions being carried out in the supercritical or near-critical fluid phase

(76) Inventors: Leo Ernest Manzer, Wilmington, DE (US); Keith W. Hutchenson, Lincoln University, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/270,011

(22) Filed: Nov. 9, 2005

Related U.S. Application Data
(60) Provisional application No. 60/626,688, filed on Nov. 10, 2004.

(51) Int. Cl. C07D 307/32

(52) U.S. Cl. 549/313

(57) ABSTRACT

Process for the production of γ-methyl-α-methylene-γ-butyrolactone from reaction of levulinic acid and hydrogen with recycle of unreacted levulinic acid and reaction of crude γ-valerolactone and formaldehyde, both reactions being carried out in the supercritical or near-critical fluid phase.

### CROSS-REFERENCE TO RELATED APPLICATION

This application claims priority under 35 U.S.C. §119 from U.S. Provisional Application Ser. No. 60/626,688, filed Nov. 10, 2004.

### FIELD OF INVENTION

This invention relates to an integrated, two-step process for producing gamma-methyl-alpha-methylene-gamma-butyrolactone (MeMBL) in a supercritical or near-critical fluid phase. The first step of the process generally involves contacting levulinic acid (LA) with hydrogen in a supercritical or near-critical fluid phase that produces gamma-valerolactone (GVL). Unreacted levulinic acid is recycled to the first step. In the second step, the resulting crude GVL, containing some residual unreacted levulinic acid, is contacted with formaldehyde in a supercritical or near-critical fluid phase in a second reactor to produce a product that contains MeMBL.

### BACKGROUND

It is known that levulinic acid can be reacted in the liquid phase with hydrogen in the presence of a suitable catalyst to produce a product that contains gamma-valerolactone (hereinafter “GVL”). See U.S. Pat. No. 6,617,464 B2. It is also known that GVL can be reacted with formaldehyde in a supercritical or near-critical fluid phase in the presence of a suitable catalyst to produce a reaction product that contains gamma-methyl-alpha-methylene-gamma-butyrolactone (hereinafter “MeMBL”). See published U.S. application 2003/0166949 A1. Generally, the first reaction step would be operated to a reasonably high levulinic acid conversion, and the product of this reaction step would be reduced in pressure and subjected to a separation process known in the art, such as vacuum distillation, to produce a high-purity (i.e., greater than about 99 mol %) GVL for use as a reactant in the second reaction step of the process, i.e., conversion of GVL to MeMBL. It might be expected that the use of crude, i.e., unpurified, GVL as a reactant to make MeMBL would result in compromised yields of MeMBL, possibly because of the presence of trace impurities, such as, perhaps, residual acid, that could deactivate or otherwise adversely affect the catalyst used to convert the GVL into MeMBL.

### SUMMARY OF THE INVENTION

Surprisingly, it has now been found that GVL containing up to about 5 mol % levulinic acid relative to GVL and levulinic acid can be used as the feed to the second reaction step of the process, without compromising the conversion of GVL to MeMBL beyond a modest initial decrease. This finding permits one to separate GVL from levulinic acid using a relatively simple separation process consisting of only a partial pressure reduction of the reaction product of the first reaction step, causing the reaction product to separate into two streams: (1) a liquid phase, containing a major portion of any unreacted levulinic acid, that can be recycled to the first reaction step, and (2) a low density phase of lower density than the liquid phase, containing GVL and a minor portion of any unreacted levulinic acid. The use of only a partial pressure reduction to effect this separation is economically preferable to a more conventional separation process (as described above): lower capital cost results from the use of relatively simple equipment (e.g., a flash tank rather than a vacuum distillation column and associated equipment), and lower variable cost results from the need for only a partial repressurization of both streams (1) and (2).

More specifically, the present invention, in its broadest embodiment, is a process for preparing gamma-methyl-alpha-methylene-gamma-butyrolactone (MeMBL), comprising the steps of:

(a) forming in a first reactor a first reaction mixture comprising levulinic acid, hydrogen and a solvent, in the presence of a first catalyst capable of converting the levulinic acid to gamma valerolactone (GVL), at a first temperature and a first pressure sufficient to cause the first reaction mixture to exist as a supercritical or near-critical fluid phase in contact with the first catalyst, thereby forming a first reaction product comprising GVL, any unreacted hydrogen, and any unreacted levulinic acid;

(b) decreasing the pressure of the first reaction product by an amount sufficient to cause the first reaction product to separate into (i) a first liquid phase comprising a major portion of the unreacted levulinic acid, and

(ii) a first low density phase, less dense than the first liquid phase, said first low density phase comprising the solvent, a major portion of the GVL, no more than about 5 mol % unreacted levulinic acid relative to the total of GVL and unreacted levulinic acid, and any unreacted hydrogen;

(c) separating the first liquid phase from the first low density phase to produce a separated first low density phase and a separated first liquid phase;

(d) introducing into a second reactor containing a second catalyst capable of converting GVL into MeMBL, the separated first low density phase, without separating any unreacted levulinic acid therefrom, and a formaldehyde source capable of forming formaldehyde, thereby forming in said second reactor a second reaction mixture at a second temperature and a second pressure sufficient to cause the second reaction mixture to exist as a supercritical or near-critical fluid phase in contact with the second catalyst, thereby forming a second reaction product comprising MeMBL, said second catalyst comprising a silica support and at least one element selected from the group consisting of potassium, cesium and rubidium.

### BRIEF DESCRIPTION OF THE DRAWING

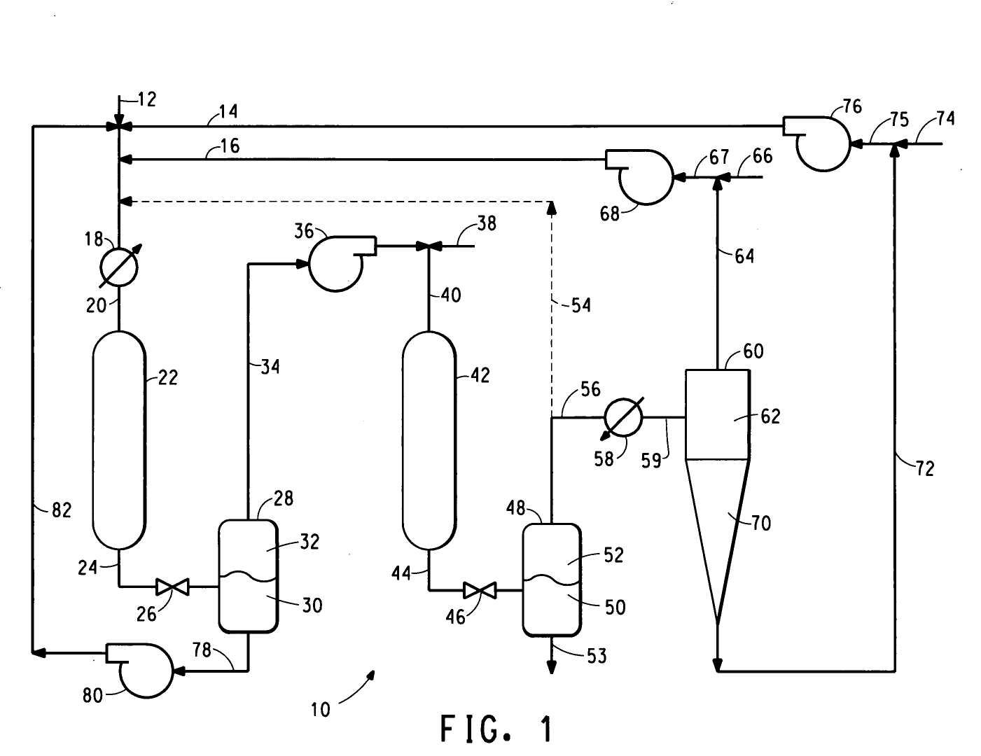

The Drawing consists of FIG. 1, which depicts a preferred embodiment of the present invention in schematic form.

### DETAILED DESCRIPTION OF THE INVENTION

Referring now to FIG. 1, there is shown in schematic form apparatus 10 for carrying out the process of the present invention as a continuous process. The present invention may also be carried out as a batch process, in which case references to “streams” should be interpreted as fixed amounts of materials introduced or otherwise treated as a single batch.

A stream 12 of levulinic acid, a stream 14 of solvent and a stream 16 of hydrogen are combined to form a stream 18 of a first reaction mixture. The hydrogen should be in molar excess relative to the levulinic acid. The first reaction mixture is pressurized and then heated in heater 20 to cause the first reaction mixture to exist as a supercritical or near-critical fluid phase, which is introduced into first reactor 22, containing a first catalyst (not shown) capable of converting the levulinic acid and hydrogen into gamma-valerolactone. In addition, the temperature of first reactor 22 should be high enough to cause the chemical reaction to proceed with desired kinetics.

Suitable first reactors 22 include fixed bed, trickle bed, and autoclave (batch or continuous stirred tank) reactors. The reactor should be configured to provide for adequate mixing of the hydrogen, levulinic acid and catalyst.

Suitable catalysts in first reactor 22 include one or more elements selected from the group consisting of palladium, ruthenium, rhenium, rhodium, iridium, platinum, nickel, cobalt, copper, iron, and osmium. The catalytic element optionally can be supported on a support. The support can be in the form of powder, granules, pellets, or the like. A compound of the element also can be supported on a support. The depositing can be accomplished by a number of well-known methods. A preferred support material can be selected from the group consisting of carbon, alumina, silica, silica-alumina, silica-titania, titania, titania-alumina, barium sulfate, calcium carbonate, strontium carbonate, and various zeolites. Most preferred supports are alumina, titania and carbon. The catalysts of the present invention may optionally comprise catalyst additives and promoters that will enhance the efficiency of the catalyst. In the processes of the invention, the preferred catalytic element content range of the supported catalyst is from about 0.1% to about 20% of the supported catalyst based on catalyst weight plus the support weight. A more preferred catalytic element content range is from about 1% to about 10% of the supported catalyst. A further preferred catalytic element content range is from about 1% to about 5% of the supported catalyst.

Other suitable unsupported catalysts include the so-called Raney® catalysts. Raney® catalysts have a high surface area due to selectively leaching an alloy containing the active element(s) and a leachable element (usually aluminum). Raney® catalysts have high activity due to the higher specific area and allow the use of lower temperatures in hydrogenation reactions. The active elements of Raney® catalysts include nickel, copper, cobalt, iron, rhodium, ruthenium, rhenium, osmium, iridium, platinum, palladium, compounds thereof and combinations thereof. Promoter elements may also be added to the base Raney® elements (available from W.R. Grace & Co., Columbia Md.) listed above to affect selectivity and/or activity of the Raney® catalyst. Promoter elements for Raney® catalysts may be selected from transition elements from Groups 3 through Group 8, and Group 11 and Group 12 of the Periodic Table of the Elements. Examples of promoter elements for the Raney® based catalytic element include chromium, molybdenum, platinum, rhodium, ruthenium, osmium, and palladium, typically at about 2% by weight of the total element.

Typical factors to consider in selecting an appropriate solvent 14 for the reaction mixture include solubility of reactants and products, chemical inertness, influence on the reaction rate and selectivity, cost, and toxicity. In addition, the critical temperature must be considered when selecting a potential solvent for conducting chemical transformations in the supercritical or near-critical fluid phase. For practical applications, thermal and catalytic chemical reactions can be conducted only in a relatively narrow temperature range. Lower temperatures result in unacceptable reaction rates, and higher temperatures can result in significant selectivity and yield losses, as well as catalyst deactivation. To obtain practical solvent densities and the corresponding density-dependent properties, temperature optimization must be balanced against a general desire to operate in the vicinity of the mixture critical point of the reaction system to fully exploit the potential advantages afforded by supercritical fluid (“SCF”) operation. The phase behavior of the reaction mixture, which is strongly influenced by the solvent critical temperature, is fundamentally important in defining this operating window, so one must select a solvent to provide the desired phase behavior. The phase behavior of SCF systems can also be manipulated to control the number and composition of coexisting phases, thus controlling both reaction effects as well as the separation of products or homogeneous catalysts from the reaction mixture. Finally, the addition of co-solvents can be effectively utilized to exploit specific solute interactions, such as enhancing solute solubilities and influencing reaction selectivities, and equilibria.

A reason often cited for using SCF-mediated reaction processes is the potential for utilizing a reaction medium that exhibits improved safety, health, and environmental impact relative to typical organic solvents. Carbon dioxide, in particular, is generally considered environmentally benign, nontoxic, nonflammable, and inexpensive, and it is suitable for use as a SCF solvent at relatively moderate temperatures. However, there are a variety of other practical SCF solvents that potentially have better solubility characteristics than CO₂ as well as beneficial impact relative to conventional liquid organic solvents.

Any suitable SCF solvent may be used in the process of this invention. A nonlimiting list of possible solvents include carbon dioxide, sulfur hexafluoride, fluoromethane, trifluoromethane, tetrafluromethane, ethane, propane, butane, isobutane, pentane, hexane, cyclohexane, water, and mixtures thereof, provided that it is inert to all reagents and products. Preferred SCF solvents include carbon dioxide or at least one C1 to C6 alkane, optionally substituted with Cl, F or Br.

The term “supercritical fluid” means a state of matter for a substance or a mixture of substances that exists above the critical temperature and critical pressure of the substance or mixture. For pure substances, the critical temperature and pressure are the highest at which vapor and liquid phases can coexist. Above the critical temperature, a liquid does not form for a pure substance, regardless of the applied pressure. Similarly, the critical pressure and critical molar volume are defined at this critical temperature corresponding to the state at which the vapor and liquid phases merge. Similarly, although more complex for multicomponent mixtures, the mixture critical state is identified as the condition at which the properties of coexisting vapor and liquid phases become indistinguishable. For a discussion of supercritical fluids, see Kirk-Othmer Encycl. of Chem. Technology, 4^{th} Ed., Vol. 23, pg. 452-477.

One of the primary advantages of SCF reaction media is that the density can be varied continuously from liquid-like to gas-like values by either varying the temperature or pressure, and to a first approximation, the solvent strength of the SCF media can be related to this continuously-variable solution density. The various density-dependent physical properties (e.g., solute solubility) also exhibit similar continuous variation in this region. In general, a SCF in the vicinity of its critical point has a liquid-like density and solvent strength, but exhibits transport properties (mass, momentum, and thermal diffusivities) that are intermediate to those of gases and liquids.

Since gaseous reactants are completely miscible with SCFs, their concentrations in SCF reaction media are significantly higher than are obtainable in conventional liquid solvents, even at appreciable pressures. These higher reactant concentrations in SCF media combined with increased component diffusivities and relatively low system viscosities can result in mass transfer rates that are appreciably higher than in liquid solvents. This can potentially shift a chemical reaction rate from mass transfer control to kinetic control in a reactor. The solubility of gaseous reactants in liquid solvents can also be enhanced by a volume expansion of the solvent with a dense supercritical fluid, which likewise results in increased mass transfer rates. Improved mass transport can also result in enhanced removal of residual solvents.

In practice, a number of desirable properties characteristic of the SCF state are realized in the expanded liquid region that exists at temperatures and pressures slightly below this critical point. A solution in this expanded liquid region is termed a “near-critical fluid” when the fluid is either at or below the critical temperature and the physical properties begin to approach those of a supercritical fluid. For the purposes of this invention, the term “near-critical fluid” includes those conditions where the fluid is at temperatures from about 75% of the critical temperature to about 100% of the critical temperature, and pressures from about 25% of the critical pressure to about 100% of the critical pressure.

The fluid state of the reaction mixture at any time during the course of a reaction is a function of the temperature, pressure, and composition. In practical applications of conducting reactions in the supercritical or near-critical fluid state, one selects an operating temperature, pressure, and mixture composition that corresponds to the desired supercritical or near-critical fluid state for the feed composition. This state may change during the course of the reaction as reactants are converted to products. For example, a reaction mixture in the SCF state may remain in the SCF state over the course of a reaction, or may undergo a phase transition to the near-critical fluid state. Conversely, a reaction mixture in the near-critical fluid state may remain in the near-critical fluid state over the course of a reaction, or may undergo a phase transition to the SCF state.

For practical applications of conducting chemical reactions in the supercritical or near-critical fluid state, one must determine the phase behavior of the mixture at the reaction conditions. One can visually observe the phase behavior of the reaction mixture by conducting the reaction in a vessel equipped with a transparent window, or by simulating the reaction mixture with a solution of similar concentration in such a vessel. Systematic determination of the phase boundaries of the reaction mixture can be determined by standard techniques using such a vessel that is also equipped with a means of varying the vessel volume at fixed composition and temperature. The vessel is loaded with the various components at the specified composition of the reaction mixture and heated to the reaction temperature. Then, the solution pressure is varied by changing the vessel volume until a phase transition is visually observed. After measuring the phase boundary of a solution of interest over the range of anticipated compositions, one can define the operating conditions (temperature and pressure) necessary to achieve the supercritical or near-critical state for conducting the desired reaction. For further discussion on experimentally determining fluid phase boundaries for a substance, see M. A. McHugh and V. J. Krukonis, Supercritical Fluid Extraction: Principles and Practice, 2^{nd} Ed., Butterworth-Heinemann: Boston (1994), pp. 85-98.

The temperature of the reaction mixture in first reactor 22 should be high enough to cause the chemical reaction to proceed with desired kinetics.

In addition, the temperature and pressure of first reactor 22 should be chosen to keep the first reaction mixture as a supercritical or near-critical fluid phase in contact with the first catalyst.

The temperature, pressure, catalyst loading and contact time can be selected to achieve a desired conversion of levulinic acid to GVL, forming a first reaction product that contains GVL, unreacted hydrogen, any unreacted levulinic acid, and solvent. A high degree of levulinic acid conversion (e.g., greater than about 95%) would be desired with a fresh catalyst to minimize the required recovery and recycle of levulinic acid downstream of the first reactor 22. However, as the catalyst deactivates with time on stream, lower conversions may be realized, which would require separation and recycle of a portion of the unreacted levulinic acid to help avoid compromising the conversion of GVL to MeMBL in the second reaction step beyond a modest initial decrease. One can determine the appropriate combination of conditions to achieve the desired level of conversion by running independent experiments in the reactor 22 that one chooses to employ in the method of the present invention.

A stream 24 of first reaction product which is withdrawn from first reactor 22 and introduced into a pressure regulator 26 to decrease the pressure of the first reaction product by an amount sufficient to cause the first reaction product to separate into two phases that are collected in separator 28: (i) a first liquid phase 30 comprising a major portion of any unreacted levulinic acid, and (ii) a first low density phase 32, less dense than the first liquid phase, comprising the solvent, a major portion of the GVL, no more than about 5 mol % unreacted levulinc acid (relative to the total of GVL and unreacted levulinic acid), and any unreacted hydrogen. The pressure in separator 28 is set at a level to effect the separation described while at the same time minimizing the magnitude of the pressure drop to avoid excessive repressurization costs. This pressure must be determined empirically with phase behavior measurements, as described above, to ensure that no more than about 5 mol % unreacted levulinc acid (relative to the total of GVL and unreacted levulinic acid) is present in first low density phase 32.

A stream 78 of the first liquid phase 30 is repressurized and recycled by pump 80 as stream 82 back to first reactor 22.

A stream 34 of first low density phase 32 is pressurized by pump 36, combined with a stream 38 of a “formaldehyde source” to form a stream 40 of a second reaction mixture, which is then introduced into second reactor 42, containing a second catalyst (not shown) capable of converting GVL into MeMBL.

The temperature of the reaction mixture in second reactor 42 should be high enough to cause the chemical reaction to proceed with desired kinetics. In addition, the temperature and pressure of second reactor 42 should be chosen to keep the second reaction mixture as a supercritical or near-critical fluid phase in contact with the second catalyst (not shown).

A “formaldehyde source” is a material that is capable of forming formaldehyde under the conditions present for the reaction in the second reactor 42 of the present method, i.e., the conversion of GVL into MeMBL. Suitable formaldehyde sources include, but are not limited to, aqueous formalin, anhydrous formaldehyde, formaldehyde hemiacetal, a low molecular weight polyformaldehyde (paraformaldehyde), or formaldehyde trimer (trioxane). The use of paraformaldehyde, trioxane, or anhydrous formaldehyde is preferred since this reduces the need to remove water from the process. Hemiacetals work effectively, but require separate steps to release formaldehyde from the alcohol and to recover and recycle the alcohol.

Suitable second catalysts comprise a silica support and at least one element selected from the group consisting of potassium, rubidium, and cesium. The silica support optionally may be doped with aluminum, zirconium and/or titanium. These catalysts preferably contain from 0.1 to 40 wt % of the catalytic element relative to the combined weight of the support plus the element (as opposed to the compound of which the element is a part). Preferably the silica-supported catalyst is porous and has a pore size distribution such that pores having a diameter between 65 and 3200 Angstroms contribute a pore volume of at least 0.3 cubic centimeters per gram of catalyst. This requirement can be ascertained by using mercury or nitrogen porosimetry.

After a suitable contact time with the second catalyst, the second reaction mixture will form a MeMBL-containing second reaction product, a stream 44 of which is withdrawn from second reactor 42 and introduced into a pressure regulator 46 to decrease its pressure to cause it to leave the supercritical or near-critical phase and form in separator 48 a second liquid phase 50 and a “second low density phase” 52 of lower density than second liquid phase 50. The second liquid phase 50 will comprise a major portion of the MeMBL produced in second reactor 42, as well as any unreacted GVL, and any unreacted formaldehyde. The second low density phase 52 will contain the solvent and any unreacted hydrogen.

A stream 53 of the second liquid phase is removed from separator 48 and the separated stream can be treated to isolate the MeMBL contained therein. Suitable methods for accomplishing the isolation include methods known in the art for separating MeMBL from unreacted GVL and formaldehyde source. A particularly suitable method for separating MeMBL from unreacted GVL involves polymerizing the MeMBL in the GVL solution using standard free radical polymerization, followed by precipitation of the poly-MeMBL, followed by thermal decomposition of the poly-MeMBL back to monomeric MeMBL. Another effective method is liquid/liquid extraction.

A stream 54 of the second low density phase 52 is removed from separator 48 to form a separated second low density phase that can be fed as stream 54 back to first reactor 22 through heater 18, or can be fed as stream 56 to cooling device 58 to produce stream 59, which can be introduced into a cyclone-type separator 60 to cause stream 59 to separate into third liquid phase 70 and gas phase 62. Third liquid phase 70 will contain primarily solvent, and gas phase 62 will contain primarily hydrogen.

A stream 64 of gas phase 62 is combined with a stream 66 of “make-up” hydrogen, and the resulting combined stream 67 is pumped by pump 68 as stream 16 back to first reactor 22 through heater 18.

A stream 72 of third liquid phase 70 is combined with a stream 74 of “make-up” solvent, and the combined stream 75 is pumped by pump 76 as stream 14 back to first reactor 22 through heater 18.

The present process may be carried out in a batch mode, in which case the “streams” referred to above will refer to discrete quantities, or in a continuous mode.

### EXAMPLES

To simulate the effect of feeding product from the first reactor that has been depressurized to produce a low density phase material containing GVL and unreacted levulinic acid to the second reactor, a series of synthetic liquid feed samples containing varying amounts of levulinic acid were prepared and fed to a reactor (corresponding to second reactor 42), as described below. The feed samples were pressurized and heated to reaction conditions prior to their being introduced into the reactor. These experiments were conducted in a continuous fixed bed reactor consisting of a 0.38-inch o.d.×0.049-inch wall×11.5-inch long 316 stainless steel tube packed with catalyst. The reactor was heated by electrical band heaters mounted around an aluminum block enclosing the reactor. The reactor was charged with 2.0 g of 20% Rb/Engelhard KA-160 SiO₂ granular catalyst. The reactant feed solution consisted of about 38 wt % gamma-valerolactone, with varying relative concentrations of levulinic acid as noted in Table 1, 2.0 wt % diphenyl ether as an internal standard, with the balance being made up with an ethanol hemiacetal solution as the formaldehyde source. The ethanol hemiacetal was prepared by refluxing a 50 mol % paraformaldehyde solution in ethanol for four hours at 95° C. followed by cooling to room temperature and filtration. This solution resulted in a 2:1 ratio of formaldehyde to gamma-valerolactone in the reactor feed, which was metered at a rate resulting in a weight hour space velocity (WHSV) in the reactor of 0.65 g gamma-valerolactone/(g catalyst-h). CO₂ was used as the solvent, and the flow rate was metered independently to give a final total organic concentration (not including CO₂) of 5 mol % in the reactor feed. The reactor was operated at a temperature of 250° C. and a pressure of about 20 MPa. The corresponding reaction profiles showing conversion of gamma-valerolactone to MeMBL are summarized in Table 1.

TABLE 1

| | GVL | GVL | GVL | GVL | GVL |
|---|---|---|---|---|---|
| | Conv. % | Conv. % | Conv % | Conv % | Conv % |
| Run | with 0 | with 1 | with 2 | with 4 | with 8 |
| Time | mol % | mol % | mol % | mol % | mol % |
| (h) | LA* | LA* | LA* | LA* | LA* |
| 0.9 | — | — | 76.3 | — | — |
| 1.0 | — | — | — | 79.5 | 58.0 |
| 1.1 | 85.7 | — | 75.6 | — | — |
| 1.3 | 84.3 | — | 76.0 | 76.8 | 57.3 |
| 1.5 | 85.0 | — | 76.0 | 76.4 | 55.4 |
| 1.7 | — | 72.5 | 76.4 | 76.7 | 51.7 |
| 2.0 | 87.9 | 73.6 | 76.4 | 76.7 | 44.6 |
| 2.3 | 90.0 | 74.1 | 76.0 | 75.8 | — |

*Molar LA concentration is relative to total GVL plus LA.

The data in Table 1 show that although there may be a modest deactivation of the catalyst used to convert the GVL to MeMBL if levulinic acid is present in the reaction mixture, the deactivation is independent of the levulinic acid concentration up to about 5 mol % relative to the combined GVL and levulinic acid. Little variation in GVL conversion is observed for residual LA feed compositions of 1, 2, and 4 mol %. However, at a LA feed composition of 8 mol %, significantly reduced GVL conversion is observed, indicating unacceptably reduced catalyst activity. Therefore, if the GVL-containing feed to the second reactor contains no more than about 5 mol % unreacted levulinic acid, then this feed material can be used directly, without separation of unreacted levulinic acid therefrom, as a reactant in the GVL to MeMBL conversion reaction. Although the data in Table 1 show the effect of levulinic acid on the catalytic activity of a rubidium-based catalyst, based on the similar catalytic activity of cesium, potassium, and rubidium for the conversion of GVL to MeMBL, as disclosed in U.S. 2003/0166949 A1, a similar result should be achievable with cesium and potassium-based catalysts in the present invention.

1. A process for preparing gamma-methyl-alpha-methylene-gamma butyrolactone (MeMBL), comprising the steps of:
(a) forming in a first reactor a first reaction mixture comprising levulinic acid, hydrogen and a solvent, in the presence of a first catalyst capable of converting the levulinic acid to gamma-valerolactone (GVL), at a first temperature and a first pressure sufficient to cause the first reaction mixture to exist as a supercritical or near-critical fluid phase in contact with the first catalyst, thereby forming a first reaction product comprising GVL, any unreacted hydrogen, and any unreacted levulinic acid;
(b) decreasing the pressure of the first reaction product by an amount sufficient to cause the first reaction product to separate into (i) a first liquid phase comprising a major portion of the unreacted levulinic acid, and (ii) a first low density phase, less dense than the first liquid phase, said first low density phase comprising the solvent, a major portion of the GVL, no more than about 5 mol % unreacted levulinic acid relative to the total of GVL and unreacted levulinic acid, and any unreacted hydrogen;
(c) separating the first liquid phase from the first low density phase to produce a separated first low density phase and a separated first liquid phase;
(d) introducing into a second reactor containing a second catalyst capable of converting GVL into MeMBL, the separated first low density phase, without separating any unreacted levulinic acid therefrom, and a formaldehyde source capable of forming formaldehyde, thereby forming in said second reactor a second reaction mixture at a second temperature and a second pressure sufficient to cause the second reaction mixture to exist as a supercritical or near-critical fluid phase in contact with the second catalyst, thereby forming a second reaction product comprising MeMBL, said second catalyst comprising a silica support and at least one element selected from the group consisting of potassium, cesium and rubidium.

2. The process of claim 1 further comprising the steps of:
(e) decreasing the second pressure of the second reaction product to cause the second reaction product to separate into (i) a second liquid phase comprising a major portion of the MeMBL, any unreacted GVL, and any unreacted formaldehyde, and (ii) a second low density phase of lower density than said second liquid phase, said second low density phase comprising the solvent and any unreacted hydrogen; and
(f) separating the second low density phase from the second liquid phase to produce a separated second low density phase.

3. The process of claim 2 further comprising the step of either (1) introducing at least a portion of the separated second low density phase into the first reactor, or (2) cooling the separated second low density phase to produce (i) a third liquid phase comprising the solvent, and (ii) a gas phase comprising the unreacted hydrogen, and (3) separating the third liquid phase from the gas phase to produce a separated third liquid phase and a separated gas phase.

4. The process of claim 3 further comprising introducing at least a portion of the separated gas phase into the first reactor.

5. The process of claim 3 further comprising introducing at least a portion of the separated third liquid phase into the first reactor.

6. The process of claim 3 further comprising introducing into the first reactor at least a portion of the separated gas phase and at least a portion of the separated third liquid phase.

7. The process of claim 1 wherein the solvent is selected from the group consisting of carbon dioxide and at least one C1 to C6 alkane, optionally substituted with Cl, F, or Br.
